(19) United States
(12) Plant Patent Application Publication
(10) Pub. No.: US 2026/0305505 P1
(43) Pub. Date: Oct. 1, 2026

(54) Partridge Berry Plant Named 'BTC-101'

(50) ;

(71) Applicant: George Barrett, Augusta, GA (US)

(72) Inventor: George Barrett, Augusta, GA (US)

(21) Appl. No.: 19/570,255

(22) Filed: Mar. 18, 2026

Related U.S. Application Data
(60) Provisional application No. 63/834,529, filed on Mar. 27, 2025.

(51) Int. Cl. A01H 6/76; A01H 5/02; A01H 5/08

(52) U.S. Cl. CPC A01H 6/76; A01H 5/02; A01H 5/08
USPC PLT/263.1

(57) ABSTRACT

A new and distinct partridge berry plant that provides a thick and complete groundcover with numerous green leaves.

### CROSS REFERENCE TO RELATED APPLICATION(S)

This application claims the benefit of priority from U.S. Provisional Patent Application No. 63/834,529, filed Mar. 27, 2025, which is incorporated herein by reference in its entirety.

Botanical classification: Mitchella repens L.

Varietal denomination: ‘BTC-101’.

### BACKGROUND OF THE INVENTION

The present invention comprises a new and distinct variety of partridge berry plant, botanically classified as Mitchella repens L. and having the varietal denomination of ‘BTC-101’. The new variety was discovered in 2020 in Augusta, Georgia in a cultivated area and selected for being the best of the seedling batch to provide an improved, evergreen groundcover. The parental varieties are unknown and the variety was first asexually reproduced in 2021 in Augusta, Georgia by cuttings and division. ‘BTC-101’ has been trial and field tested and has been found to retain its distinctive characteristics and remain true to type through successive propagations. The present invention has not been evaluated under all possible environmental conditions. The phenotype may vary with variations in environment without a change in the genotype of the plant.

‘BTC-101’ has a refined growth habit. It is an evergreen, non-climbing vine that has dark green foliage, red berries, and white flowers. The variety grows very low, hugging the ground, and provides at least 2.5 cm in thickness of a complete cover over the ground. The fullness of the provided groundcover of ‘BTC-101’ distinguishes it from other partridge berry varieties known to the breeder. Its evergreen nature is similar to other partridge berry varieties known to the breeder. When compared to partridge berry plant named ‘BTC-102’, ‘BTC-102’ differs in growth habit and new growth having a lighter green leaf color than ‘BTC-101’.

### DESCRIPTION OF THE DRAWINGS

The accompanying photographic drawings taken from 12-18 months of age illustrate the new variety, with the colors being as nearly true as is possible with color illustrations of this type.

FIG. 1 is a close-up view of the foliage and a berry of ‘BTC-101’;

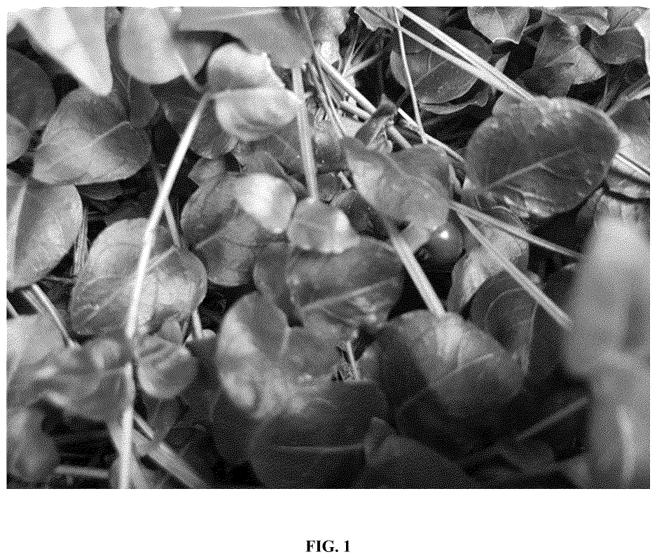

FIG. 2 illustrates the foliage of ‘BTC-101’; and

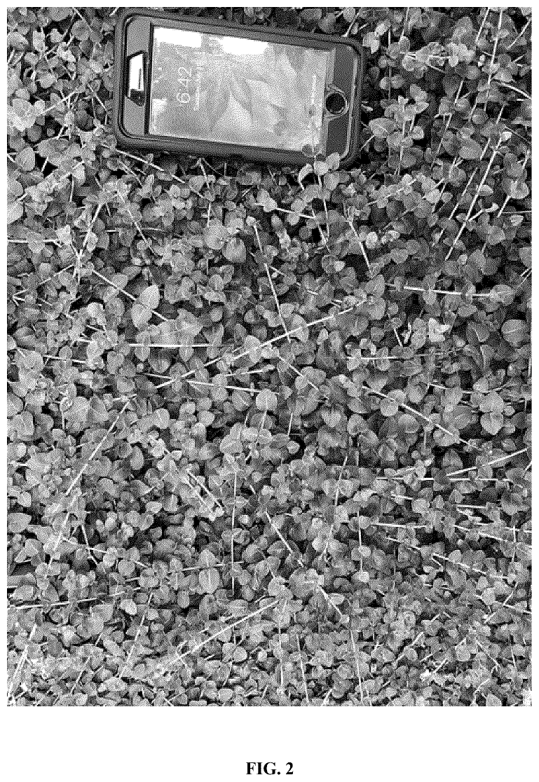

FIG. 3 illustrates the flowers of ‘BTC-101’.

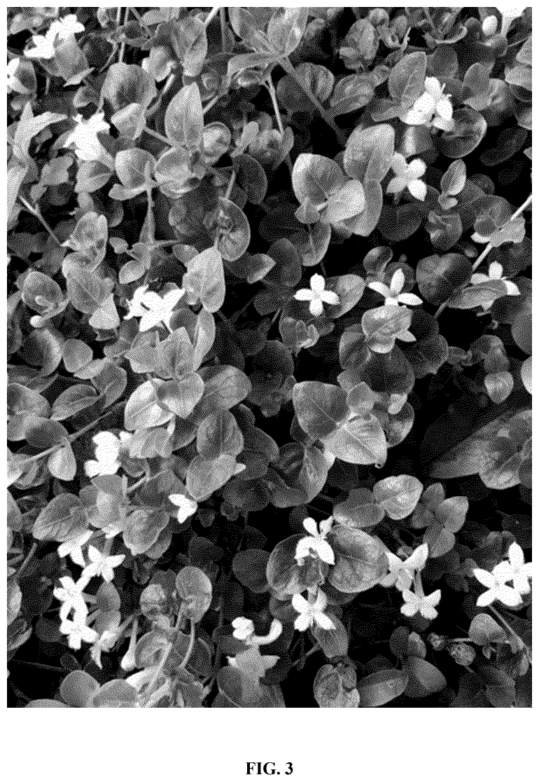

### DESCRIPTION OF THE PLANT

The following detailed description sets forth the characteristics of the new variety at approximately 18 months of age. The data which defines these characteristics was collected under natural light in Augusta, Georgia. Plants of the new variety were grown in 1020 propagation trays. Color references are primarily to the Hex color codes available on www.htmlcolorcodes.com and www.color-hex.com, except where general color terms are used.

### PLANT

Time to initiate roots: About 30 days at about 33-37° C.
Time to produce a rooted young plant: About 60-90 days at about 30-35° C.
Root description: Fibrous—each node roots, with a color of FFEFB6.
Growth habit: Trailing and spreading vine.
Plant height (soil level to top of plant): 5.0-10.0 cm.
Plant width: Potentially infinite, depending on the size of the growth area.
Disease resistance/susceptibility: None observed to date.
Pest resistance/susceptibility: None observed to date.
Temperature tolerance: No damage observed in USDA Hardiness Zone 8b temperatures.
Drought tolerance: High.
Stems:
   Average number per plant.—Vine.
   Average number of stems per node.—1.
   Length.—Variable, stem lengthens throughout the growing season.
   Diameter.—10.0-20.0 mm.
   Color.—Young stems: FFD0C0. Mature stems: 644A35.
Petioles:
   Length.—0.8 cm.
   Diameter.—Approximately 10.0 mm.
   Texture.—Smooth.
   Angle.—45° out of stem and then curves into leaf base.
Leaves:
   Arrangement.—Opposite; simple.
   Average number per plant.—Numerous — too many to quantify.
   Average number per stem.—Numerous — too many to quantify.
   Length.—2.0 cm.
   Width.—1.9 cm.
   Shape of leaf (generally).—Cordiform; widely ovate.
   Shape of apex.—Acute.
   Shape of base.—Slightly cordiform.
   Margin description.—Entire.
   Texture.—Upper surface: Smooth. Lower surface: Smooth.
   Aspect.—Flat — parallel with the ground.
   Pubescence.—None present.
   Color.—Young leaves: Upper surface: A4DBAD. Lower surface: C9ECBE. Mature leaves: Upper surface: DOC8B6. Lower surface: 59623D.
   Veins.—Venation pattern: Pinnate. Color: Upper surface: 49572A. Lower surface: 5F6839.

### FLOWERS/BERRIES

(Not available at the time of specification preparation):
Flower arrangement: Paired flowers atop peduncles.
Flower number per plant: Numerous—too many to quantify.
Flower timing at a specific location: April/May to June in Augusta, Georgia.
Overall description: 4-ovate-lobed funnelform flowers.
Petal color: White with a sightly pink hue.

### REPRODUCTIVE ORGANS

(Not available at the time of specification preparation):

I claim:

1. A new and distinct variety of partridge berry plant named ‘BTC-101’, botanically known as Mitchella repens L., as is herein illustrated and described.
